(19) United States
(12) Patent Application Publication
(10) Pub. No.: US 2021/0217495 A1
(43) Pub. Date: Jul. 15, 2021

(54) Method For Machine Learning To Find Patterns In Ensembles Of Biological Sequences Based On Biophysical Properties

(71) Applicant: Beth Israel Deaconess Medical Center, Inc., Boston, MA (US)

(72) Inventors: Ramy Arnaout, Chestnut Hill, MA (US); Rohit Arora, Braintree, MA (US); Joseph John Kaplinsky, London (GB)

(21) Appl. No.: 17/058,795

(22) PCT Filed: Jun. 24, 2019
(86) PCT No.: PCT/US2019/038660

§ 371 Date: Nov. 25, 2020

Related U.S. Application Data
(60) Provisional application No. 62/693,252, filed on Jul. 2, 2018.

(51) Int. Cl. G16B 40/20; G16B 5/20; G16B 20/40

(52) U.S. Cl. CPC G16B 40/20; G16B 20/40; G16B 5/20

(57) ABSTRACT

A computer-implemented system and method for associating immune system repertoires with specific stimuli (exposures) based on the biophysical properties of the repertoire's receptors. Sequences of a training repertoire are converted into a set of biophysical properties, and a computer-based compact representation of the training repertoire is built using maximum entropy modeling. In one version, an “immunome-wide association study” is performed by computer scoring a test repertoire using several such models to classify the test repertoire as being associated with a biological condition or not. In another version, one or more sets of parameters from the models are found that together classify each model as being from an individual that has the condition or from an individual that does not.

### RELATED APPLICATION

This application claims the benefit of U.S. Provisional Application No. 62/693,252, filed on Jul. 2, 2018. The entire teachings of the above application are incorporated herein by reference.

### GOVERNMENT SUPPORT

This invention was made with government support under Grant No. AI114958 awarded by the National Institutes of Health. The government has certain rights in the invention.

### BACKGROUND

Exposure to infectious agents results in expansion of specific B- and T-cell clones, characterized by epitope-specific antibody and T-cell receptor (TCR) genes. High-throughput single-cell repertoire sequencing can be used to determine the frequency of each clone in a blood sample, but not which expanded clones are specific to a given exposure, as opposed to past/intercurrent exposures, or to bystander activation. If the specific clones were known, their presence and frequency could be used to detect specific exposures. Because clonal expansion is a signal amplifier, such an approach to diagnosis may be more sensitive than direct pathogen detection, especially in chronic infections such as, for example, tuberculosis (TB), in which the immune response plays a prominent role in disease pathogenesis, and in infections such as Lyme disease, in which the organism is difficult to grow in the laboratory. If the specific clones were known for many different exposures, those exposures could be diagnosed simultaneously from a single sequence-based test. In addition to diagnostic utility, the sequences that define specific clones could be explored as potential reagents and therapeutics. This is the promise of high-throughput repertoire sequencing.

The challenge is to determine which clones are specific to a given exposure. One strategy is to sequence repertoires from many exposed individuals to identify sequences that are seen more often than by chance, using repertoires from unexposed individuals as controls. However, the diversity of TCR and especially antibody sequences means that repertoires from different exposed individuals rarely contain the same expanded clones, necessitating very large cohorts.

There is, therefore, an ongoing need to provide automated techniques of finding patterns in, and of classifying, immune repertoires.

### SUMMARY

In accordance with an embodiment of the invention, there is provided a computer-based system and method for associating immune system repertoires with specific stimuli (exposures) based on the biophysical properties of the repertoire's receptors. Sequences of a training repertoire are converted into a set of biophysical properties, and a computer-based compact representation of the training repertoire is built using maximum entropy modeling. In one version, an “immunome-wide association study” is performed by computer scoring a test repertoire using several such models to classify the test repertoire as being associated with a biological condition or not. In another version, one or more sets of parameters from the computer-based models are found that together classify each model as being from an individual that has the condition or from an individual that does not.

In one embodiment according to the invention, there is provided a computer-implemented method of classifying an immune system repertoire. The computer-implemented method comprises providing a data structure representing a plurality of training biological sequences that are included in at least one training immune system repertoire; and, for the training biological sequences represented by the data structure, associating, in a manner automated by a processor, one or more biophysical properties and operatively indicating the biophysical properties in a plurality of training repertoire biophysical feature data structures. The training repertoire biophysical feature data structures computationally represent the one or more biophysical properties of the training biological sequences based on expectation values of at least one biophysical composite measure for each of a plurality of feature components. The plurality of feature components includes feature components corresponding to an amino acid sequence of the training biological sequences. A maximum entropy model is formed, in an automated fashion by the digital processor, based on the training repertoire biophysical feature data structures. The formed maximum entropy model comprises a bias parameter for each feature component of the plurality of feature components. A data structure is provided representing a plurality of test biological sequences that are included in at least one test immune system repertoire. Based on the formed maximum entropy model and the data structure representing the plurality of test biological sequences, the test immune system repertoire is classified, in an automated fashion by the processor. The classifying includes classifying the test immune system repertoire as being associated with at least one biological condition or as not being associated with the at least one biological condition.

In further, related embodiments, classifying the test immune system repertoire may comprise scoring, in an automated fashion by the processor, the data structure representing the plurality of test biological sequences against both (i) at least one biological condition-positive maximum entropy model determined based on a training repertoire biophysical feature data structure that is known to be associated with the at least one biological condition, and (ii) at least one biological condition-negative maximum entropy model determined based on a training repertoire biophysical feature data structure that is known not to be associated with the at least one biological condition. The method may further comprise forming, in an automated fashion by the processor, an all-model score classifier module implemented by the processor, the forming of the all-model score classifier module comprising determining with the processor a plurality of all-model scores against both the at least one biological condition-positive maximum entropy model and the at least one biological condition-negative maximum entropy model. The all-model classifier module may permit generating, in an automated fashion by the processor, data structures representing at least one of: a histogram of the plurality of all-model scores versus a fraction of the test biological sequences, and a two or more dimensional cloud of the all-model scores. Forming the all-model score classifier module may comprise dividing, in an automated fashion by the processor, the plurality of scores against the at least one biological condition-positive maximum entropy model by the plurality of scores against the at least one biological condition-negative maximum entropy model, the dividing comprising desired weighting and normalizing. The computer-based method may further comprise classifying, in an automated fashion by the processor, the test immune system repertoire based on an increased probability density beyond expected probability density determined based on at least a portion of at least one of: the data structure representing the histogram of the plurality of all-model scores, and the data structure representing the two or more dimensional cloud of the all-model scores.

In other related embodiments, classifying the test immune system repertoire may comprise determining, in an automated fashion by the processor, a reduced subset of the bias parameters of the maximum entropy model that permit classifying the test immune system repertoire with a desired level of accuracy as being systematically associated with, or not systematically associated with, the at least one biological condition. The reduced subset of bias parameters may be determined in an automated fashion by the processor based at least on the bias parameters of the maximum entropy model using a Metropolis-Hastings Markov-Chain Monte-Carlo procedure. The reduced subset of bias parameters may be determined, in an automated fashion by the processor, based at least on the bias parameters of the maximum entropy model, using at least one of a principal component analysis procedure, an independent component analysis procedure, a maximum accuracy separator module, such as a linear support-vector machine classifier, or other cost-minimizing procedure, implemented in an automated fashion by the processor, to separate at least one biological condition-positive maximum entropy model from at least one biological condition-negative maximum entropy model.

In further related embodiments, the at least one biophysical composite measure may comprise a result of a dimensionality reduction of a plurality of individual amino acid measures. The dimensionality reduction may comprise at least one of: a principal components analysis dimensionality reduction, an independent components analysis dimensionality reduction, a t-distributed stochastic neighbor embedding dimensionality reduction, a non-negative matrix factorization dimensionality reduction, a linear discriminant analysis dimensionality reduction, a generalized discriminant analysis dimensionality reduction, and an autoencoder dimensionality reduction. The plurality of individual amino acid measures may comprise physical measures and chemical measures of each of twenty naturally-occurring amino acids, or of at least one artificial amino acid. The at least one biophysical composite measure may comprise ten or fewer biophysical composite measures. The plurality of feature components may further include a plurality of feature components corresponding to at least one of: nearest neighbor pairs of the amino acid sequence of the training biological sequences; next-nearest neighbor pairs of the amino acid sequence of the training biological sequences; third-nearest neighbor pairs of the amino acid sequence of the training biological sequences; fourth-nearest neighbor pairs of the amino acid sequence of the training biological sequences; symmetric cross pairs of the amino acid sequence of the training biological sequences; asymmetric cross pairs of the amino acid sequence of the training biological sequences; amino acid triples of the amino acid sequence of the training biological sequences; a complementarity-determining region length distribution of the amino acid sequence of the training biological sequences; consecutive quadruples of amino acids of the amino acid sequence of the training biological sequences; at least one stem property of the amino acid sequence of the training biological sequences; at least one loop property of the amino acid sequence of the training biological sequences; and at least one complementarity-determining region property of the amino acid sequence of the training biological sequences.

In other, related embodiments, the training biological sequences may comprise at least one of antibodies and T-cell receptors, and may comprise both antibodies and T-cell receptors. The at least one biological condition may comprise at least one of: a vaccination, an infection, an autoimmune condition, a disease, a transfusion reaction, a transplant rejection, aging, a cancer, a gender, a geographical background and a species, strain or genotype. The method may further comprise determining, in an automated fashion by the processor, a probability of the test immune system repertoire having been generated by the maximum entropy model. The method may further comprise determining, in an automated fashion by the processor, similarity scores comparing at least two different test immune system repertoires with each other based on the maximum entropy model, or similarity scores comparing at least two different sequences with each other based on the maximum entropy model. Forming the maximum entropy model may comprise training, in an automated fashion by the processor, the maximum entropy model on the plurality of feature components using a Metropolis-Hastings Markov-Chain Monte-Carlo procedure.

In another embodiment according to the invention, there is provided a computer-implemented method of generating a biological sequence data structure corresponding to an immune system repertoire, using a maximum entropy model previously generated by: providing a data structure representing a plurality of training biological sequences that are included in at least one training immune system repertoire; for the training biological sequences represented by the data structure, associating, in a manner automated by a processor, one or more biophysical properties and operatively indicating the biophysical properties in a plurality of training repertoire biophysical feature data structures; the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences; and forming, in an automated fashion by the processor, a maximum entropy model based on the training repertoire biophysical feature data structures, the formed maximum entropy model comprising a bias parameter for each feature component of the plurality of feature components. The computer-implemented method comprises, based on a maximum entropy model so determined, forming, in an automated fashion with a processor, a new biological sequence data structure representing an immune system repertoire comprising similar biophysical properties to the at least one training immune system repertoire, based on at least the bias parameters of the maximum entropy model.

In another embodiment according to the invention, there is provided a computer system for classifying an immune system repertoire. The computer system comprises a training sequence module configured to provide, in a manner automated by a processor, a data structure representing a plurality of training biological sequences that are included in at least one training immune system repertoire. A feature translator module is configured to associate, for the training biological sequences represented by the data structure, in a manner automated by a processor, one or more biophysical properties and to operatively indicate the biophysical properties in a plurality of training repertoire biophysical feature data structures. The training repertoire biophysical feature data structures computationally represent the one or more biophysical properties of the training biological sequences based on expectation values of at least one biophysical composite measure for each of a plurality of feature components, the plurality of feature components including feature components corresponding to an amino acid sequence of the training biological sequences. A modeling module is configured to form, in an automated fashion by the processor, a maximum entropy model based on the training repertoire biophysical feature data structures, the formed maximum entropy model comprising a bias parameter for each feature component of the plurality of feature components. A test sequence module is configured to provide, in a manner automated by a processor, a data structure representing a plurality of test biological sequences that are included in at least one test immune system repertoire. A classifier module is configured to, based on the formed maximum entropy model and the data structure representing the plurality of test biological sequences, classify, in an automated fashion by the processor, the test immune system repertoire, the classifying including classifying the test immune system repertoire as being associated with at least one biological condition or as not being associated with the at least one biological condition.

In further, related embodiments, the classifier module may be further configured to classify the test immune system repertoire by scoring, in an automated fashion by the processor, the data structure representing the plurality of test biological sequences against both (i) at least one biological condition-positive maximum entropy model determined based on a training immune system repertoire that is known to be associated with the at least one biological condition, and (ii) at least one biological condition-negative maximum entropy model determined based on a training immune system repertoire that is known not to be associated with the at least one biological condition. The system may further comprise an all-model score generator configured to form, in an automated fashion by the processor, an all-model score classifier module implemented by the processor, the forming of the all-model score classifier module comprising determining with the processor a plurality of all-model scores against both the at least one biological condition-positive maximum entropy model and the at least one biological condition-negative maximum entropy model. The all-model classifier module may permit generating, in an automated fashion by the processor, a data structure representing at least one of: a histogram of the plurality of all-model scores versus a fraction of the test biological sequences, and a two or more dimensional cloud of the all-model scores. The all-model score generator may be further configured to form the all-model score classifier by dividing, in an automated fashion by the processor, the plurality of scores against the at least one biological condition-positive maximum entropy model by the plurality of scores against the at least one biological condition-negative maximum entropy model, the dividing comprising desired weighting and normalizing. The classifier module may be further configured to classify, in an automated fashion by the processor, the test immune system repertoire based on an increased probability density beyond expected probability density determined based on at least a portion of at least one of: the data structure representing the histogram of the plurality of all-model scores, and the data structure representing the two or more dimensional cloud of the all-model scores.

In other, related embodiments, the classifier module may be further configured to classify the test immune system repertoire based on determining, in an automated fashion by the processor, a reduced subset of the bias parameters of the maximum entropy model that permit classifying the test immune system repertoire with a desired level of accuracy as being systematically associated with, or not systematically associated with, the at least one biological condition. The classifier module may be further configured to determine the reduced subset of bias parameters, in an automated fashion by the processor, based at least on the bias parameters of the maximum entropy model using a Metropolis-Hastings Markov-Chain Monte-Carlo procedure. The classifier module may be further configured to determine the reduced subset of bias parameters, in an automated fashion by the processor, based at least on the bias parameters of the maximum entropy model using at least one of a principal component analysis procedure, an independent component analysis procedure, a linear support-vector machine classifier, or other cost-minimizing procedure. The system may further comprise a maximum accuracy separator module configured to separate, in an automated fashion by the processor, at least one biological condition-positive maximum entropy model from at least one biological condition-negative maximum entropy model. The maximum accuracy separator module may comprise a linear support-vector machine classifier.

In further related embodiments, the at least one biophysical composite measure may comprise a result of a dimensionality reduction of a plurality of individual amino acid measures. The dimensionality reduction may comprise at least one of: a principal components analysis dimensionality reduction, an independent components analysis dimensionality reduction, a t-distributed stochastic neighbor embedding dimensionality reduction, a non-negative matrix factorization dimensionality reduction, a linear discriminant analysis dimensionality reduction, a generalized discriminant analysis dimensionality reduction and an autoencoder dimensionality reduction. The plurality of individual amino acid measures may comprise physical measures and chemical measures of each of twenty naturally-occurring amino acids, and may comprise at least one artificial amino acid. The at least one biophysical composite measure may comprise ten or fewer biophysical composite measures. The plurality of feature components may further include a plurality of feature components corresponding to at least one of: nearest neighbor pairs of the amino acid sequence of the training biological sequences; next-nearest neighbor pairs of the amino acid sequence of the training biological sequences; third-nearest neighbor pairs of the amino acid sequence of the training biological sequences; fourth-nearest neighbor pairs of the amino acid sequence of the training biological sequences; symmetric cross pairs of the amino acid sequence of the training biological sequences; asymmetric cross pairs of the amino acid sequence of the training biological sequences; amino acid triples of the amino acid sequence of the training biological sequences; a complementarity-determining region length distribution of the amino acid sequence of the training biological sequences; consecutive quadruples of amino acids of the amino acid sequence of the training biological sequences; at least one stem property of the amino acid sequence of the training biological sequences; at least one loop property of the amino acid sequence of the training biological sequences; and at least one complementarity-determining region property of the amino acid sequence of the training biological sequences.

In other related embodiments of the computer system, the training biological sequences may comprise at least one of antibodies and T-cell receptors, such as both antibodies and T-cell receptors. The at least one biological condition may comprise at least one of: a vaccination, an infection, an autoimmune condition, a disease, a transfusion, a transplant, aging, a cancer, a gender, a geographical background and a species, strain or genotype. The classifier module may further comprise a probability determination module configured to determine, in an automated fashion by the processor, a probability of the test immune system repertoire having been generated by the maximum entropy model. The classifier module may be further configured to determine, in an automated fashion by the processor, similarity scores comparing at least two different test immune system repertoires with each other based on the maximum entropy model. The modeling module may be configured to form the maximum entropy model by training, in an automated fashion by the processor, the maximum entropy model on the plurality of feature components using a Metropolis-Hastings Markov-Chain Monte-Carlo procedure.

In another embodiment according to the invention, there is provided a non-transitory computer-readable medium configured to store instructions for classifying an immune system repertoire. The instructions, when loaded and executed by a processor, cause the processor to classify the immune system repertoire by: providing a data structure representing a plurality of training biological sequences that are included in at least one training immune system repertoire; for the training biological sequences represented by the data structure, associating, in a manner automated by a processor, one or more biophysical properties and operatively indicating the biophysical properties in a plurality of training repertoire biophysical feature data structures; the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences; forming, in an automated fashion by the processor, a maximum entropy model based on the training repertoire biophysical feature data structures, the formed maximum entropy model comprising a bias parameter for each feature component of the plurality of feature components; providing a data structure representing a plurality of test biological sequences that are included in at least one test immune system repertoire; and based on the formed maximum entropy model and the data structure representing the plurality of test biological sequences, classifying, in an automated fashion by the processor, the test immune system repertoire, the classifying including classifying the test immune system repertoire as being associated with at least one biological condition or as not being associated with the at least one biological condition.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic block diagram of an immune system repertoire classification system, in accordance with an embodiment of the invention.

FIG. 2 is a schematic block diagram illustrating operation of a classifier module, in accordance with an embodiment of the invention.

FIG. 3 is a schematic block diagram illustrating operation of an all-model score generator module and an all-model score classifier module, in accordance with an embodiment of the invention.

FIG. 4 is a schematic block diagram illustrating operation of a classifier module to produce a reduced subset of bias parameters, in accordance with an embodiment of the invention.

FIG. 5 is a schematic block diagram illustrating operation of a maximum accuracy separator module, in accordance with an embodiment of the invention.

FIG. 6 is a schematic block diagram of a method of dimensionality reduction to produce a biophysical composite measure, in accordance with an embodiment of the invention.

FIG. 7 is a flow diagram of a computer-implemented method of classifying an immune system repertoire, in accordance with an embodiment of the invention.

FIG. 8 is a flow diagram of a computer-implemented method of generating a biological sequence data structure corresponding to an immune system repertoire, using a previously-generated maximum entropy model, in accordance with an embodiment of the invention.

FIG. 9 illustrates a computer network or similar digital processing environment in which embodiments of the present invention may be implemented.

FIG. 10 is a diagram of an example internal structure of a computer (e.g., client processor/device or server computers) in the computer system of FIG. 9.

### DETAILED DESCRIPTION

A description of example embodiments follows.

The adaptive immune system consists of B cells, which make antibodies, and T cells, which target infected or cancerous cells. Its power is in being able to respond to almost any stimulus. As a result it plays important roles in many conditions and health interventions, including vaccination, infection, autoimmunity, cardiovascular disease, transfusion, transplant, aging, and cancer. The key to this breadth is that each new B or T cell makes a unique receptor—the antibody in B cells and the T-cell receptor in T cells—that targets that cell to specific parts of specific molecules, called antigens or epitopes, related to the stimulus. When cells encounter their stimuli, they divide, producing more cells with their specific receptors. Repeat encounters thus result in a signal related to this specific stimulus, which should be detectable against the background of non-specific B- and T-cells that make up the rest of the B- and T-cell repertoire. The primary obstacle to detection is that there are so many receptors that a person's cells can make—in fact, orders of magnitude more than the number of B or T cells that a person actually has—that the same stimulus may stimulate different receptors in different people. Thus, there may be no common signal at the level of detail of receptors' nucleotide or amino-acid sequences. Yet because receptors work though binding, i.e. through having shapes complementary to their antigens or epitopes, it is reasonable to expect a signal in the biophysical properties that determine the shapes of receptors, which receptors that differ at the sequence level may share.

In accordance with an embodiment of the invention, there is provided a method for associating B- and T-cell repertoires with specific stimuli based on the biophysical properties of the repertoire's receptors. Specifically, a machine-learning approach of maximum entropy modeling is used that: (i) takes as input a list of antibody or T-cell receptor sequences (a “training repertoire”); (ii) converts each sequence into a set of biophysical properties; and then (iii) builds a compact representation (“model”) of the training repertoire that can be used to score a test repertoire. In one embodiment (iv), referred to as an immunome-wide association study (IWAS), the test repertoire is scored by several models, some of which were trained on repertoires from individuals who have a certain condition (e.g. cytomegalovirus [CMV] infection) and others of which were trained on repertoires from individuals who do not have that condition (e.g. uninfected controls), to classify the test repertoire as being associated with that condition or not. In a second embodiment, (v) one or more sets of parameters from the models are found that together (including through transformations such as principal components analysis or independent components analysis) classify each model as being from an individual that has the condition or from an individual that does not.

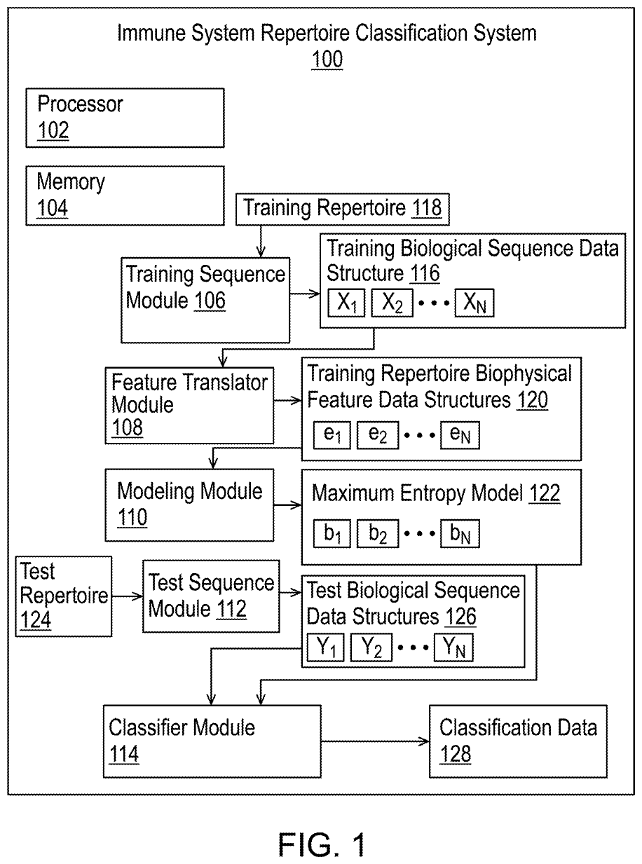

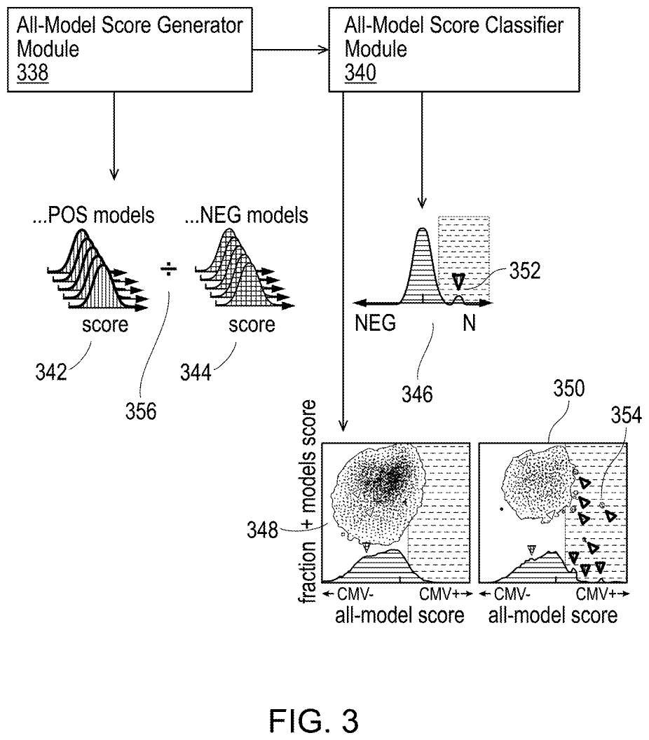

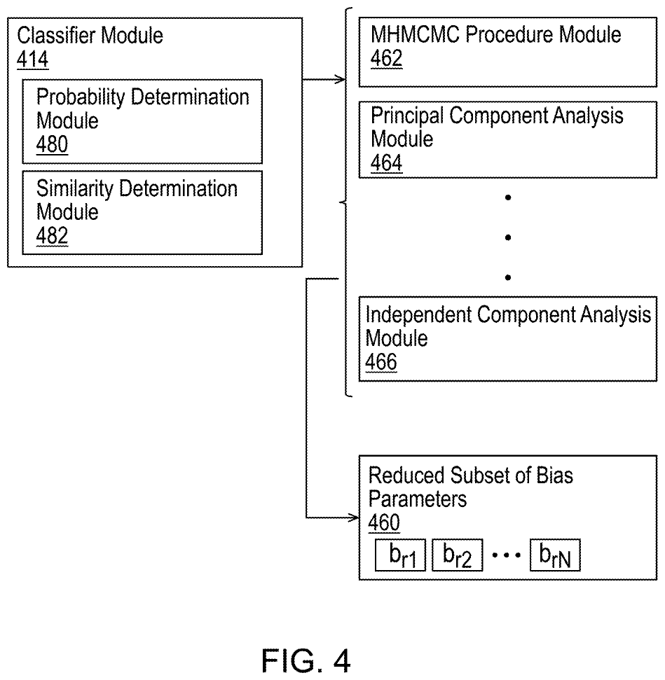

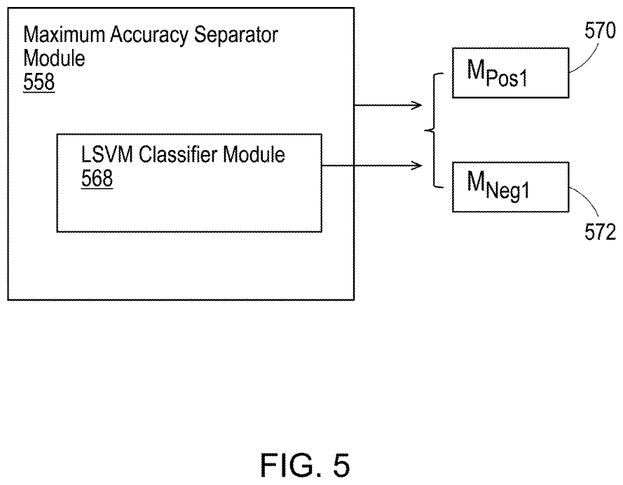

FIG. 1 is a schematic block diagram of an immune system repertoire classification system 100, in accordance with an embodiment of the invention. The computer system 100 includes a processor 102 and a memory 104, which stores computer code instructions. The processor 102 and the memory 104, with the computer code instructions, are configured to implement: a training sequence module 106, a feature translator module 108, a modeling module 110, a test sequence module 112 and a classifier module 114. In addition, in other embodiments according to the invention, the processor 102 and memory 104 may be configured to implement one or more of: an all-model score generator module 338 and an all-model score classifier module 340 (see FIG. 3); a probability determination module 480 and similarity determination module 482, a Metropolis-Hastings Markov-Chain Monte-Carlo (MHMCMC) procedure module 462, a principal component analysis module 464, an independent component analysis module 466 (see FIG. 4); a maximum accuracy separator module 558, which may be a Linear Support-Vector Machine Classifier Module (LSVM) or other score-minimizing module 568 (see FIG. 5). It will be appreciated that processor 102 and memory 104 may be implemented on one or more separate processors and one or more separate memories, any combination of which cooperate together to implement all or a portion of embodiments herein.

In the embodiment of FIG. 1, the computer system 100 comprises a training sequence module 106 configured to provide, in a manner automated by processor 102, a data structure 116 representing a plurality of training biological sequences that are included in at least one training immune system repertoire 118. The training biological sequences included in the training repertoire 118 may comprise at least one of antibodies and T-cell receptors, such as both antibodies and T-cell receptors. A feature translator module 108 is configured to associate, for the training biological sequences represented by the data structure 116, in a manner automated by processor 102, one or more biophysical properties and to operatively indicate the biophysical properties in a plurality of training repertoire biophysical feature data structures 120. The training repertoire biophysical feature data structures 120 computationally represent the one or more biophysical properties of the training biological sequences based on expectation values, indicated as e₁, e₂ . . . e_{N} in data structures 120, of at least one biophysical composite measure for each of a plurality of feature components. The plurality of feature components including feature components corresponding to an amino acid sequence of the training biological sequences, and may include other feature components. For example, the plurality of feature components may further include a plurality of feature components corresponding to at least one of: nearest neighbor pairs of the amino acid sequence of the training biological sequences; next-nearest neighbor pairs of the amino acid sequence of the training biological sequences; third-nearest neighbor pairs of the amino acid sequence of the training biological sequences; fourth-nearest neighbor pairs of the amino acid sequence of the training biological sequences; symmetric cross pairs of the amino acid sequence of the training biological sequences; asymmetric cross pairs of the amino acid sequence of the training biological sequences; amino acid triples of the amino acid sequence of the training biological sequences; a complementarity-determining region length distribution of the amino acid sequence of the training biological sequences; consecutive quadruples of amino acids of the amino acid sequence of the training biological sequences; at least one stem property of the amino acid sequence of the training biological sequences; at least one loop property of the amino acid sequence of the training biological sequences; and at least one complementarity-determining region property of the amino acid sequence of the training biological sequences.

In the embodiment of FIG. 1, a modeling module 110 is configured to form, in an automated fashion by the processor 102, a maximum entropy model 122 based on the training repertoire biophysical feature data structures 120. For example, the modeling module 110 can be configured to form the maximum entropy model 122 by training, in an automated fashion by the processor 102, the maximum entropy model 122 on the plurality of feature components using a Metropolis-Hastings Markov-Chain Monte-Carlo procedure. The formed maximum entropy model 122 comprises a bias parameter, indicated as b₁, b₂ . . . b_{N} in maximum entropy model 122, for each feature component of the plurality of feature components. A test sequence module 112 is configured to provide, in a manner automated by processor 102, a data structure representing a plurality of test biological sequences that are included in at least one test immune system repertoire 124. A classifier module 114 is configured to, based on the formed maximum entropy model 122 and the data structure 126 representing the plurality of test biological sequences, classify, in an automated fashion by the processor 102, the test immune system repertoire 124, which can produce classification data 128. The classifying includes classifying the test immune system repertoire 124 as being associated with at least one biological condition or as not being associated with the at least one biological condition, which can be indicated in the classification data 128 in one or more data structures. The classification data 128 can, for example, be a display indicator, a data feed as input to another program, a signal to another device/controller/software application, or other kinds of processor output of classification data 128. Although examples of immune repertoires resulting from infections are referred to herein, it should be appreciated that, as used herein, a “biological condition” can comprise at least one of: a vaccination, an infection, an autoimmune condition, a disease, a transfusion, a transplant, aging, a cancer, a gender, a geographical background and a species, strain or genotype.

In accordance with an embodiment of the invention, the maximum entropy model 122 is composed of a set of parameters called biases, indicated as b₁, b₂ . . . b_{N} in the maximum entropy model 122 of FIG. 1. Together, these biases describe how a given repertoire differs from a collection of random sequences. Each bias corresponds to a feature of the repertoire that was measured, where the features have been chosen prior to measurement, as part of feature selection, which is the first step in the machine-learning algorithm. For example, the average charge of amino acids in the repertoire might be measured. Then the model 122 would include a bias that corresponds to the average charge. If it were desired to construct a given repertoire, using the model 122 as a set of instructions, and if the average-charge bias has a positive value in the model 122, then there would be a bias towards choosing more positively charged amino acids in constructing the repertoire. Thus, the bias can be thought of as a “finger on the scale” that pushes one away from choosing amino acids at random, and toward (in this case) choosing amino acids with positive charge. Once features have been chosen and measured, maximum-entropy modeling is used to find these biases.

It should be noted that, in accordance with an embodiment of the invention, the bias for a feature differs from the measurement of the feature in the training repertoire 118. For datasets as complex as immune repertoires, describing them requires many features; for example, a model in accordance with an embodiment of the invention can contain on the order of 10³ to 10⁴ features.

In accordance with an embodiment of the invention, one or more sets of parameters from the models are found that together (including through transformations such as principal components analysis or independent components analysis) classify each model as being from an individual that has a biological condition or from an individual that does not. Together, the biases that comprise a given model describe a given repertoire. Exposure to an infection, e.g. cytomegalovirus (CMV), will result in changes to the sequence composition of a repertoire. The biases describe this composition (indeed, the biases can be used to generate a repertoire that is statistically indistinguishable from the repertoire, and in this sense, as a shorthand, the model is a generative model that can re-create its repertoire). Therefore, exposure affects the biases. However, all sorts of other interpersonal differences will also affect the biases, so some of the biases will differ systematically between people exposed to e.g. CMV and people who are not, and other biases will differ randomly between those people. In accordance with an embodiment of the invention, the subset of biases is found that differ systematically. (For example, we might find that in CMV bias #1 is >0.32, bias #2 is ≤0.4, bias #3 is either less than 0.4 or greater than 1.2, etc.). This subset is used as a classifier to classify unknown repertoires' models as being positive or negative for a biological condition, e.g. CMV+ or CMV−. In an investigation of cytomegalovirus (CMV) performed in accordance with an embodiment of the invention, an independent-components analysis (ICA) was performed to reduce a set of 5-20 biases to two dimensions. It was found that a linear support-vector machine classifier (the line that best separates CMV+from CMV− models) gives an uncorrected accuracy of ˜90% relative to the existing gold-standard test (serology). However, because the gold standard itself is imperfect, with an accuracy of 92-97% depending on the study, the corrected accuracy of an embodiment according to the invention was found to be also 92-97%—as good as the industry standard.

An embodiment according to the invention applies to finding patterns in any ensemble of biological sequences based on biophysical patterns.

In accordance with an embodiment of the invention, the training repertoire biophysical data structures 120 are based on biophysical properties, instead of on amino acids or nucleotide sequences. There are many fewer features (each requiring a parameter that must be fit) than conventional techniques. For example, conventional methods that modeled 20 amino acids required 20 parameters (19 independent) to represent amino-acid frequencies, another 400 (399 independent) to represent nearest-neighbor amino-acid pairs, yet another 400 for next-nearest-neighbor pairs, and so on. Accurate sampling of large numbers of features requires impractically large training sets. In contrast, in one embodiment, an embodiment according to the invention uses five parameters for amino-acid properties (which themselves summarize dozens of specific biophysical measurements) in place of the 20 for amino-acid frequencies, resulting in 5×5=25 for nearest-neighbor pairs, another 25 for next-nearest-neighbors, and so forth. This allows an embodiment according to the invention to consider more features (e.g. more distant pairs; sets of three or four positions) with training sets that are sufficiently small to usefully be derived from clinical samples. Models in accordance with an embodiment of the invention can easily generate new sequences that have similar biophysical properties to those in training repertoires. This includes generation of sequences that have similar properties to multiple training repertoires (e.g. from different infections), and that differ from multiple others (e.g. autoimmune diseases), simultaneously. An embodiment according to the invention outputs the probability of each sequence being generated by a model or set of models. (The sum of probabilities for all sequences equals 1.) Having probabilities makes it possible to calculate relative probabilities that any given sequence is consistent with one or another repertoire, which is potentially useful for generating candidate sequences with desired properties. In addition, an embodiment according to the invention permits various similarity scores between different repertoires, e.g. repertoires from two different people or over time, which may be useful for discovering new relationships with various health conditions.

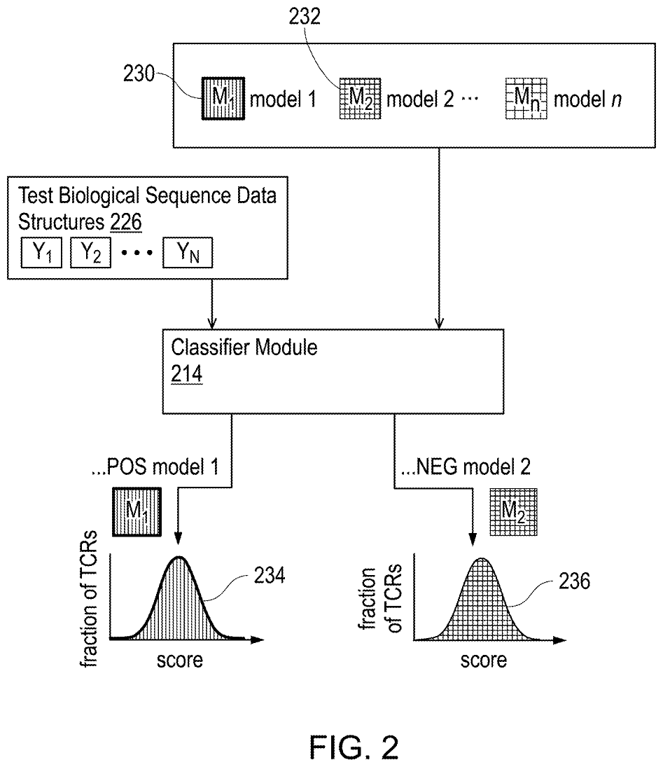

FIG. 2 is a schematic block diagram illustrating operation of a classifier module 214 (like 114 of FIG. 1), in accordance with an embodiment of the invention. The classifier module 214 is configured to classify the test immune system repertoire (124, of FIG. 1) using scoring performed in an automated fashion by the processor (102, of FIG. 1). In the embodiment of FIG. 2, the data structure 226 representing the plurality of test biological sequences is scored against both (i) at least one biological condition-positive maximum entropy model 230, determined based on a training immune system repertoire that is known to be associated with the at least one biological condition, and (ii) at least one biological condition-negative maximum entropy model 232, determined based on a training immune system repertoire that is known not to be associated with the at least one biological condition. The resulting score against the at least one condition-positive model 230 can, for example, be data indicative of a histogram 234 of a fraction of T-cell receptors or other sequences that have scores in a given range against the one or more condition-positive models 230. Likewise, the resulting score against the at least one condition-negative model 232 can, for example, be data indicative of a histogram 236 of a fraction of T-cell receptors or other sequences that have scores in a given range against the one or more condition-negative models.

FIG. 3 is a schematic block diagram illustrating operation of an all-model score generator module 338 and an all-model score classifier module 340, in accordance with an embodiment of the invention. The system 100 (see FIG. 1) may further comprise an all-model score generator 338 configured to form, in an automated fashion by the processor 102 (see FIG. 1), an all-model score classifier module 340 implemented by the processor 102 (see FIG. 1). The forming of the all-model score classifier module 340 comprises determining with the processor 102 (see FIG. 1) a plurality of all-model scores against both the at least one biological condition-positive maximum entropy model 342 and the at least one biological condition-negative maximum entropy model 344. The all-model classifier module 340 may permit generating, in an automated fashion by the processor 102 (see FIG. 1), a data structure representing at least one of: a histogram 346 of the plurality of all-model scores versus a fraction of the test biological sequences, and a two or more dimensional cloud 348, 350 of the all-model scores. For example, a two-dimensional cloud 348 of the all-model scores against the biological-condition negative maximum entropy model and a two-dimensional cloud 350 of the all-model scores against the biological-condition positive maximum entropy model can be formed. The all-model score generator 338 may be further configured to form the all-model score classifier 340 by dividing 356, in an automated fashion by the processor 102 (see FIG. 1), the plurality of scores 342 against the at least one biological condition-positive maximum entropy model by the plurality of scores 344 against the at least one biological condition-negative maximum entropy model. The dividing 356 can comprise desired weighting and normalizing. The result of the dividing is the relative probability of each immune sequence being associated with biological condition-positive status or biological-condition negative status. The classifier module 114 (see FIG. 1) may be further configured to classify, in an automated fashion by the processor 102 (see FIG. 1), the test immune system repertoire 124 (see FIG. 1) based on an increased probability density beyond expected probability density 352, 354, determined based on at least a portion of at least one of: the data structure representing the histogram 346 of the plurality of all-model scores, and the data structure 348, 350 representing the two or more dimensional cloud of the all-model scores. For example, in the histogram 346, a right-tail spike 352 can represent an increased probability density beyond expected probability density, which can be indicative of a biological condition-positive repertoire; or, for example, in the two-dimensional cloud 350, an isolated patch 354 can likewise represent an increased probability density beyond expected probability density, which can be indicative of a biological condition-positive repertoire. In accordance with an embodiment of the invention, combining positive 342 and negative 344 model scores yields an all-model-score classifier 346, which enhances signals: sequences favored by positive models (at 352) are pushed toward the right tail. Below the two-dimensional clouds 348, 350, there are shown histograms of all-models scores on CMV− and CMV+ subjects, in an investigation conducted in accordance with an embodiment of the invention. The CMV+ pattern is seen as spikes in the right-hand tails, underneath cloud 350, which contain TCRs associated with CMV status. These are more easily seen at 354 in the 2D clouds above each histogram. Right-tail spikes are notably absent in CMV− repertoires 348. Other spikes likely represent clones not related to CMV. There are, for example, about 200,000 TCRs per plot, in plots 348 and 350.

FIG. 4 is a schematic block diagram illustrating operation of the classifier module 414 (like 114 of FIG. 1) to produce a reduced subset of bias parameters 460, in accordance with an embodiment of the invention. In this embodiment, the classifier module 414 is configured to classify the test immune system repertoire 124 (see FIG. 1) based on determining, in an automated fashion by the processor 102 (see FIG. 1), a reduced subset of the bias parameters 460 of the maximum entropy model 122 that permit classifying the test immune system repertoire 124 (see FIG. 1) with a desired level of accuracy as being systematically associated with, or not systematically associated with, the at least one biological condition. The reduced subset of the bias parameters 460 are in indicated in FIG. 4 as parameters bᵣ₁, bᵣ₂, . . . b_{rN}. For example, the classifier module 414 can be configured to determine the reduced subset of bias parameters 460, based at least on the bias parameters of the maximum entropy model 122 (see FIG. 1) using a Metropolis-Hastings Markov-Chain Monte-Carlo (MHMCMC) procedure, which can be implemented in an automated fashion by the processor 102 (see FIG. 1) using an MHMCMC module 462. Alternatively or in addition, the classifier module 414 can be configured to determine the reduced subset of bias parameters 460, in an automated fashion by the processor 102 (see FIG. 1), based at least on the bias parameters of the maximum entropy model using at least one of a principal component analysis procedure and an independent component analysis procedure, implemented respectively by a Principal Components Analysis Module 464 and an Independent Component Analysis Module 466, or other cost-minimizing procedure. In other embodiments, the classifier module 414 may further comprise a probability determination module 480 configured to determine, in an automated fashion by the processor 102 (see FIG. 1), a probability of the test immune system repertoire 124 (see FIG. 1) having been generated by the maximum entropy model 122 (see FIG. 1). The classifier module 414 may be further configured to determine similarity scores in an automated fashion by the processor 102 (see FIG. 1). For example, the classifier module 414 employs a similarity determination module 482 to generate similarity scores. The similarity scores compare at least two different test immune system repertoires 124 (see FIG. 1) with each other, based on the maximum entropy model 122 (see FIG. 1).

FIG. 5 is a schematic block diagram illustrating operation of a maximum accuracy separator module 558, in accordance with an embodiment of the invention. In this embodiment, the system 100 (see FIG. 1) includes a maximum accuracy separator module 558, configured to separate, in an automated fashion by the processor, at least one biological condition-positive maximum entropy model 570 from at least one biological condition-negative maximum entropy model 572. For example, the maximum accuracy separator module 558 may comprise a linear support-vector machine classifier 568, although it will be appreciated that other kinds of maximum accuracy separators can be used.

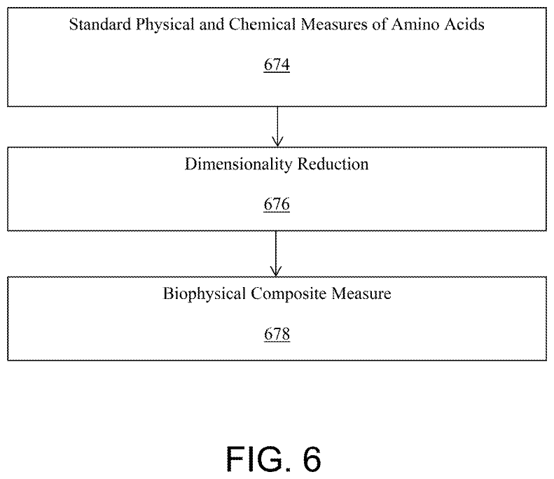

FIG. 6 is a schematic block diagram of a method of dimensionality reduction to produce a biophysical composite measure, in accordance with an embodiment of the invention. One or more standard physical and chemical measures of amino acids 674 are subjected to a dimensionality reduction 676, to produce one or more biophysical composite measures 678. The resulting biophysical composite measure 678 can be used in the training repertoire biophysical feature data structures 120 (see FIG. 1) to computationally represent the one or more biophysical properties of the training biological sequences based on expectation values, indicated as e₁, e₂ . . . e_{N} in data structures 120 (of FIG. 1), of at least one biophysical composite measure for each of a plurality of feature components. The at least one biophysical composite measure can comprise a result of a dimensionality reduction of a plurality of individual amino acid measures. In a particular example, the individual amino acid measures may be the 26 physicochemical descriptor variables identified in “New chemical descriptors relevant for the design of biologically active peptides. A multivariate characterization of 87 amino acids,” Sandberg M., et al., J Med Chem, 1998 Jul 2; 41(14):2481-91, hereinafter “Sandberg, 1998,” the entire teachings of which are hereby incorporated herein by reference. For example, the dimensionality reduction used to obtain the biophysical composite measure can comprise at least one of: a principal components analysis dimensionality reduction, an independent components analysis dimensionality reduction, a t-distributed stochastic neighbor embedding dimensionality reduction, a non-negative matrix factorization dimensionality reduction, a linear discriminant analysis dimensionality reduction, a generalized discriminant analysis dimensionality reduction and an autoencoder dimensionality reduction. The plurality of individual amino acid measures can comprise physical measures and chemical measures of each of twenty naturally-occurring amino acids, and can comprise physical measures and chemical measures of at least one artificial amino acid. The at least one biophysical composite measure may comprise ten or fewer biophysical composite measures. For example, as few as five or fewer biophysical composite measures can be used to represent the amino acids in a sequence.

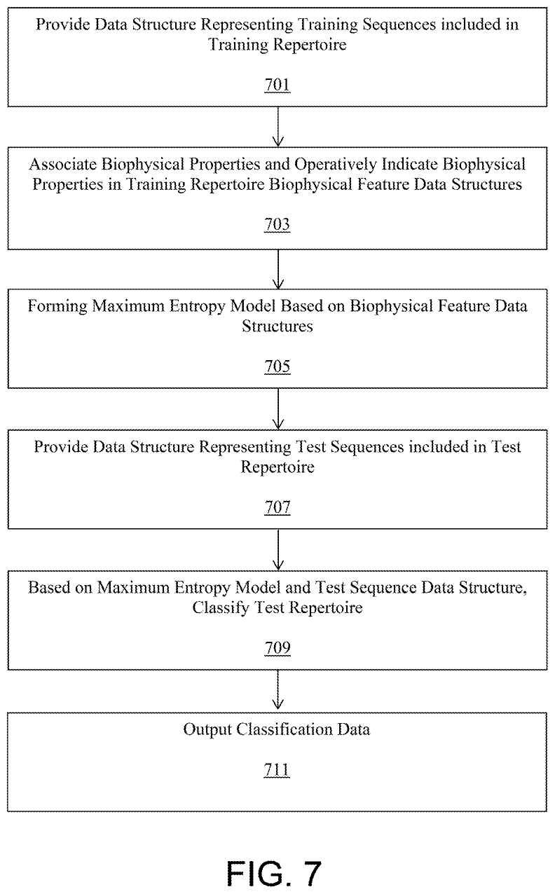

FIG. 7 is a flow diagram of a computer-implemented method of classifying an immune system repertoire, in accordance with an embodiment of the invention. The computer-implemented method comprises providing 701 a data structure representing a plurality of training biological sequences that are included in at least one training immune system repertoire; and, for the training biological sequences represented by the data structure, associating 703, in a manner automated by a processor, one or more biophysical properties and operatively indicating the biophysical properties in a plurality of training repertoire biophysical feature data structures. The training repertoire biophysical feature data structures computationally represent the one or more biophysical properties of the training biological sequences based on expectation values of at least one biophysical composite measure for each of a plurality of feature components. The plurality of feature components includes feature components corresponding to an amino acid sequence of the training biological sequences. A maximum entropy model is formed, 705, in an automated fashion by the processor, based on the training repertoire biophysical feature data structures. The formed maximum entropy model comprises a bias parameter for each feature component of the plurality of feature components. A data structure is provided, 707, representing a plurality of test biological sequences that are included in at least one test immune system repertoire. Based on the formed maximum entropy model and the data structure representing the plurality of test biological sequences, the test immune system repertoire is classified, 709, in an automated fashion by the processor. The classifying includes classifying the test immune system repertoire as being associated with at least one biological condition or as not being associated with the at least one biological condition. Classification data is output 711, for example, as a display indicator, a data feed as input to another program, a signal to another device/controller/software application, or other kinds of processor output; and can include: a data indication that the test immune system repertoire is associated with at least one biological condition or is not associated with the at least one biological condition; a data indication to assist with diagnosis of at least one biological condition, identification of a drug candidate, a therapeutic indicator, or other processor output taught herein.

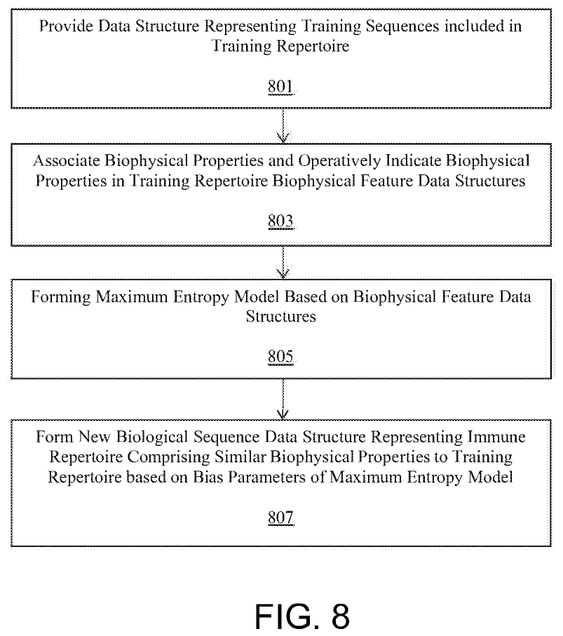

FIG. 8 is a flow diagram of a computer-implemented method of generating a biological sequence data structure corresponding to an immune system repertoire, using a previously-generated maximum entropy model, in accordance with an embodiment of the invention. The maximum entropy model is previously generated by: providing 801 a data structure representing a plurality of training biological sequences that are included in at least one training immune system repertoire; for the training biological sequences represented by the data structure, associating, 803, in a manner automated by a processor, one or more biophysical properties and operatively indicating the biophysical properties in a plurality of training repertoire biophysical feature data structures; the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences; and forming, 805, in an automated fashion by the processor, a maximum entropy model based on the training repertoire biophysical feature data structures, the formed maximum entropy model comprising a bias parameter for each feature component of the plurality of feature components. The computer-implemented method comprises, based on a maximum entropy model so determined, forming, 807, in an automated fashion with a processor, a new biological sequence data structure representing an immune system repertoire comprising similar biophysical properties to the at least one training immune system repertoire, based on at least the bias parameters of the maximum entropy model.

Techniques in accordance with an embodiment of the invention can, for example, be used to provide information that assists with diagnostics, and for therapeutics, and reagents. In diagnostics, for example, an embodiment can be used to assist with classifying a test repertoire as being consistent with certain conditions. In this use case, a repertoire is obtained from a test subject and sequenced. The repertoire is then scored by sets of models that have been trained on repertoires from subjects with various conditions. If the test repertoire scores highly, the information can be used to assist with diagnosing a test subject with that condition. Note that the test subject can be tested simultaneously for any condition for which models exist. Thus, a single test could indicate whether, e.g., the test subject's vaccinations are achieving their desired effect, whether the test subject has been exposed to or is infected with any of a wide range of agents, whether they have an immune response to cancer or cancer therapy, whether they are at risk for a transfusion reaction or transplant rejection, and whether their immune system indicates premature aging.

Other embodiments can be used for therapeutic products. For example, to identify biological drug candidates, antibodies that score well according to models of repertoires from a given viral infection would serve as candidates for a drug that could prevent or treat that infection. In another embodiment, a system and method can be used in the same way to generate potential reagents, since antibodies are a major class of reagents in biomedical research and in clinical diagnostic testing.

By contrast, for diagnosis, the presently available standard of care in the field, for most infectious and autoimmune conditions, is use of a kit that contains an antibody-based reagent that is used to stain cells in a blood or tissue sample, or a reagent (which may be cells or a protein) derived from the agent that is mixed with patient serum to detect antibodies to the agent. For leukemias, the standard of care diagnostic is flow cytometry, usually following the appearance of unusual white cells on microscopy and disturbances in counts of white-cell subsets (again on routine flow cytometry). For lymphomas and most other cancers, it is biopsy and staining, usually with antibody-based reagents, occasionally supplemented by narrow-target sequence-based testing. By contrast with such conventional techniques, an embodiment according to the invention provides (i) the ability to provide information that assists with diagnosis of many conditions in a single “universal” test and (ii) to propose many new potential candidate drugs or reagents based on biophysical properties.

As used herein, a “biological sequence” is a sequence including a protein (such as, for example, a protein of a T-cell receptor or an antibody), or a nucleic acid.

As used herein, a “protein” is a biological molecule consisting of one or more chains of amino acids. Proteins differ from one another primarily in their sequence of amino acids, which is dictated by the nucleotide sequence of the encoding gene. A peptide is a single linear polymer chain of two or more amino acids bonded together by peptide bonds between the carboxyl and amino groups of adjacent amino acid residues; multiple peptides in a chain can be referred to as a polypeptide. Proteins can be made of one or more polypeptides. Shortly after or even during synthesis, the residues in a protein are often chemically modified by posttranslational modification, which alters the physical and chemical properties, folding, stability, activity, and ultimately, the function of the proteins. Sometimes proteins have non-peptide groups attached, which can be called prosthetic groups or cofactors. It will be appreciated, in addition, that a biological sequence can include non-natural bases and residues, for example, non-natural amino acids inserted into a biological sequence.

As used herein, “nucleic acid” refers to a macromolecule composed of chains (a polymer or an oligomer) of monomeric nucleotide. The most common nucleic acids are deoxyribonucleic acid (DNA) and ribonucleic acid (RNA). It should be further understood that the present invention can be used for biological sequences containing artificial nucleic acids such as peptide nucleic acid (PNA), morpholino, locked nucleic acid (LNA), glycol nucleic acid (GNA) and threose nucleic acid (TNA), among others. In various embodiments of the present invention, nucleic acids can be derived from a variety of sources such as bacteria, virus, humans, and animals, as well as sources such as plants and fungi, among others. The source can be a pathogen. Alternatively, the source can be a synthetic organism. Nucleic acids can be genomic, extrachromosomal or synthetic. Where the term “DNA” is used herein, one of ordinary skill in the art will appreciate that the methods and devices described herein can be applied to other nucleic acids, for example, RNA or those mentioned above. In addition, the terms “nucleic acid,” “polynucleotide,” and “oligonucleotide” are used herein to include a polymeric form of nucleotides of any length, including, but not limited to, ribonucleotides or deoxyribonucleotides. There is no intended distinction in length between these terms. Further, these terms refer only to the primary structure of the molecule. Thus, in certain embodiments these terms can include triple-, double- and single-stranded DNA, PNA, as well as triple-, double- and single-stranded RNA. They also include modifications, such as by methylation and/or by capping, and unmodified forms of the polynucleotide. More particularly, the terms “nucleic acid,” “polynucleotide,” and “oligonucleotide,” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from Anti-Virals, Inc., Corvallis, Oreg., U.S.A., as Neugene) polymers, and other synthetic sequence-specific nucleic acid polymers providing that the polymers contain nucleobases in a configuration which allows for base pairing and base stacking, such as is found in DNA and RNA.

In an embodiment according to the invention, processes described as being implemented by one processor may be implemented by component processors, and/or a cluster of processors, configured to perform the described processes, which may be performed in parallel synchronously or asynchronously. Such component processors may be implemented on a single machine, on multiple different machines, in a distributed fashion in a network, or as program module components implemented on any of the foregoing.

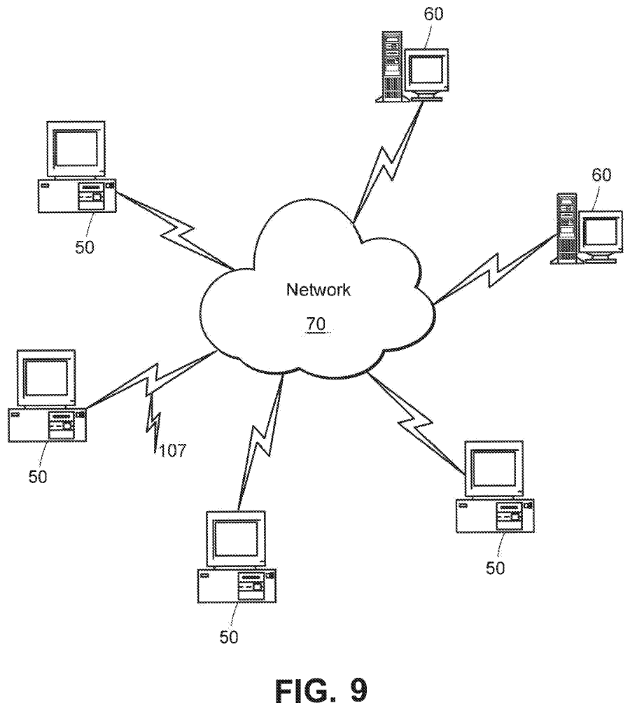

FIG. 9 illustrates a computer network or similar digital processing environment in which embodiments of the present invention may be implemented. Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

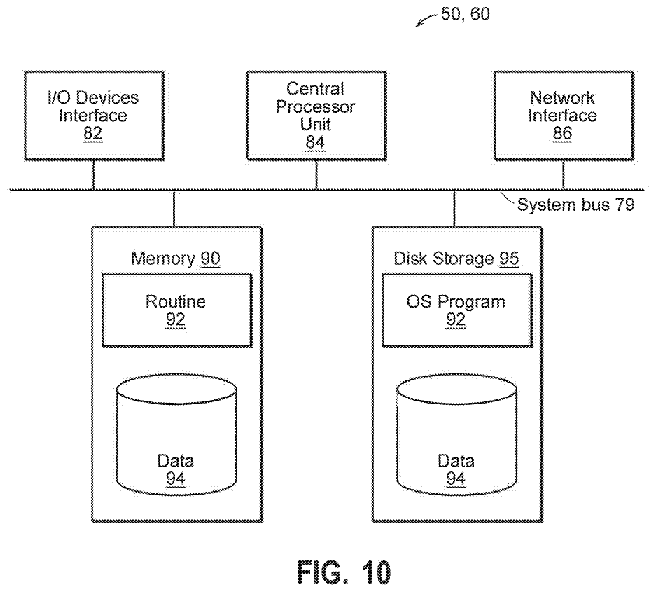

FIG. 10 is a diagram of an example internal structure of a computer (e.g., client processor/device 50 or server computers 60) in the computer system of FIG. 9. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 9). Memory 90 provides volatile storage for computer software instructions 92 and data 94 used to implement an embodiment of the present invention (including, for example, to implement one or more of: a training sequence module 106, a feature translator module 108, a modeling module 110, a test sequence module 112, a classifier module 114, an all-model score generator module 338, an all-model score classifier module 340, a probability determination module 480, a similarity determination module 482, a Metropolis-Hastings Markov-Chain Monte-Carlo (MHMCMC) procedure module 462, a principal component analysis module 464, an independent component analysis module 466, a maximum accuracy separator module 558, and a Linear Support-Vector Machine Classifier Module (LSVM) 568, detailed herein). Disk storage 95 provides non-volatile storage for computer software instructions 92 and data 94 used to implement an embodiment of the present invention. A central processor unit 84 is also attached to the system bus 79 and provides for the execution of computer instructions, for example having a flow of data and control like that of FIGS. 7 and 8.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection 107. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer.

In other embodiments, the software instructions 92 and data 94 are provided on a cloud platform, as SaaS (Software as a Service), and the like.

### Experimental:

In investigations conducted in accordance with an embodiment of the invention, directed to cytomegalovirus (CMV), for which data is shown in FIG. 3, the following techniques of feature selection, modeling and classification were used, some or all of which techniques can be used in accordance with embodiments set forth herein.

### Feature Selection:

Principal-component dimensionality reduction was performed on a set of 26 standard physical and chemical measures of each the 20 amino acids (Sandberg, 1998) to obtain five composite measures that together explained 92% of the overall variance; the top three of these corresponded roughly to side-chain surface area, size, and charge. Expectation values for each composite measure were calculated across productively recombined CDR3s of a given input repertoire. In addition, expectation values for the product for each nearest, next-nearest, third-nearest, and cross-loop pair and for each amino-acid triple, as well as for the CDR3 length distribution, were also calculated. In some experiments additional expectation values (e.g. fourth-nearest neighbor pairs, consecutive quadruples of amino acids, properties of CDRs 1 and 2, stems vs. loops) were also used.

### Modeling:

For each repertoire, a maximum-entropy model was trained on these features using a Metropolis-Hastings Markov-chain Monte-Carlo (MHMCMC) approach, testing Damerau-Levenshtein distances to confirm no autocorrelation in the sampling chains (R² >0.999 for observed vs. uncorrelated distance distributions), and with a stopping condition of the size of the sample not exceeding that of the training set to avoid overfitting. Validity was tested statistically on both toy and real repertoires by confirming decreasing root-mean-squared distance between expectation values of the training set and MHMMC samples, and functionally by confirming significant overlap in sequence identity/similarity between the training set and samples. Robustness was confirmed statistically by comparing all biases of repeat fits (R² >0.999) and confirming no outliers and empirically by confirming that the final samples from repeat fits contained similar sequences.

### Classification:

Each repertoire from an exposed individual was thought of as consisting of a disease-specific signal superimposed on background processes, with the majority of variation in model biases most likely due to background processes. A MHMCMC search was performed to find sets of biases that classified repertoires by disease status with high accuracy, using 10-fold cross-validation at each step to decrease the risk of overfitting and repeating this search on hundreds of randomly relabeled datasets to reject the null hypothesis that the resulting accuracy of such a classifier could be achieved by chance. Robustness was confirmed by repeat searches finding in the same set. To measure the expected accuracy on unseen data, exhaustive leave one-out testing was performed in which a classifier was trained on all but one model and tested on the holdout, with the accuracy measured over all tests (similar leave-n-out/explicit validation-set testing can be applied when datasets are sufficiently large).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A computer-implemented method of classifying an immune system repertoire, the computer-implemented method comprising:
providing a data structure representing a plurality of training biological sequences that are included in at least one training immune system repertoire;
for the training biological sequences represented by the data structure, associating, in a manner automated by a processor, one or more biophysical properties and operatively indicating the biophysical properties in a plurality of training repertoire biophysical feature data structures;
the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences;
forming, in an automated fashion by the processor, a maximum entropy model based on the training repertoire biophysical feature data structures, the formed maximum entropy model comprising a bias parameter for each feature component of the plurality of feature components;
providing a data structure representing a plurality of test biological sequences that are included in at least one test immune system repertoire; and
based on the formed maximum entropy model and the data structure representing the plurality of test biological sequences, classifying, in an automated fashion by the processor, the test immune system repertoire, the classifying including classifying the test immune system repertoire as being associated with at least one biological condition or as not being associated with the at least one biological condition.

2. The computer-implemented method of claim 1, wherein the classifying the test immune system repertoire comprises scoring, in an automated fashion by the processor, the data structure representing the plurality of test biological sequences against both (i) at least one biological condition-positive maximum entropy model determined based on a training repertoire biophysical feature data structure that is known to be associated with the at least one biological condition, and (ii) at least one biological condition-negative maximum entropy model determined based on a training repertoire biophysical feature data structure that is known not to be associated with the at least one biological condition.

3. The computer-implemented method of claim 2, further comprising forming, in an automated fashion by the processor, an all-model score classifier module implemented by the processor, the forming of the all-model score classifier module comprising determining with the processor a plurality of all-model scores against both the at least one biological condition-positive maximum entropy model and the at least one biological condition-negative maximum entropy model, the all-model classifier module permitting generating, in an automated fashion by the processor, data structures representing at least one of: a histogram of the plurality of all-model scores versus a fraction of the test biological sequences, and a two or more dimensional cloud of the all-model scores.

4. The computer-implemented method of claim 3, wherein forming the all-model score classifier module comprises dividing, in an automated fashion by the processor, the plurality of scores against the at least one biological condition-positive maximum entropy model by the plurality of scores against the at least one biological condition-negative maximum entropy model, the dividing comprising desired weighting and normalizing.

5. The computer-implemented method of claim 3, further comprising classifying, in an automated fashion by the processor, the test immune system repertoire based on an increased probability density beyond expected probability density determined based on at least a portion of at least one of: the data structure representing the histogram of the plurality of all-model scores, and the data structure representing the two or more dimensional cloud of the all-model scores.

6. The computer-implemented method of claim 1, wherein the classifying the test immune system repertoire comprises determining, in an automated fashion by the processor, a reduced subset of the bias parameters of the maximum entropy model that permit classifying the test immune system repertoire with a desired level of accuracy as being systematically associated with, or not systematically associated with, the at least one biological condition.

7-8. (canceled)

9. The computer-implemented method of claim 6, further comprising using a maximum accuracy separator module, implemented in an automated fashion by the processor, to separate at least one biological condition-positive maximum entropy model from at least one biological condition-negative maximum entropy model.

10. The computer-implemented method of claim 9, wherein the maximum accuracy separator module comprises a linear support-vector machine classifier.

11. The computer-implemented method of claim 1, wherein the at least one biophysical composite measure comprises a result of a dimensionality reduction of a plurality of individual amino acid measures.

12. -19. (Canceled)

20. The computer-implemented method of claim 1, further comprising determining, in an automated fashion by the processor, a probability of the test immune system repertoire having been generated by the maximum entropy model.

21. The computer-implemented method of claim 1, further comprising determining, in an automated fashion by the processor, similarity scores comparing at least two different test immune system repertoires with each other based on the maximum entropy model, or similarity scores comparing at least two different sequences with each other based on the maximum entropy model.

22. The computer-implemented method of claim 1, wherein the forming the maximum entropy model comprises training, in an automated fashion by the processor, the maximum entropy model on the plurality of feature components using a Metropolis-Hastings Markov-Chain Monte-Carlo procedure.

23. A computer-implemented method of generating a biological sequence data structure corresponding to an immune system repertoire, using a maximum entropy model previously generated by,
providing a data structure representing a plurality of training biological sequences that are included in at least one training immune system repertoire;
for the training biological sequences represented by the data structure, associating, in a manner automated by a processor, one or more biophysical properties and operatively indicating the biophysical properties in a plurality of training repertoire biophysical feature data structures;
the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences;
forming, in an automated fashion by the processor, a maximum entropy model based on the training repertoire biophysical feature data structures, the formed maximum entropy model comprising a bias parameter for each feature component of the plurality of feature components,
the computer-implemented method comprising:
based on a maximum entropy model so determined, forming, in an automated fashion with a processor, a new biological sequence data structure representing an immune system repertoire comprising similar biophysical properties to the at least one training immune system repertoire, based on at least the bias parameters of the maximum entropy model.

24. A computer system for classifying an immune system repertoire, the computer system comprising:
a training sequence module configured to provide, in a manner automated by a processor, a data structure representing a plurality of training biological sequences that are included in at least one training immune system repertoire;
a feature translator module configured to associate, for the training biological sequences represented by the data structure, in a manner automated by a processor, one or more biophysical properties and to operatively indicate the biophysical properties in a plurality of training repertoire biophysical feature data structures;
the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences;
a modeling module configured to form, in an automated fashion by the processor, a maximum entropy model based on the training repertoire biophysical feature data structures, the formed maximum entropy model comprising a bias parameter for each feature component of the plurality of feature components;
a test sequence module configured to provide, in a manner automated by a processor, a data structure representing a plurality of test biological sequences that are included in at least one test immune system repertoire; and
a classifier module configured to, based on the formed maximum entropy model and the data structure representing the plurality of test biological sequences, classify, in an automated fashion by the processor, the test immune system repertoire, the classifying including classifying the test immune system repertoire as being associated with at least one biological condition or as not being associated with the at least one biological condition.

25. The computer system of claim 24, wherein the classifier module is further configured to classify the test immune system repertoire by scoring, in an automated fashion by the processor, the data structure representing the plurality of test biological sequences against both (i) at least one biological condition-positive maximum entropy model determined based on a training immune system repertoire that is known to be associated with the at least one biological condition, and (ii) at least one biological condition-negative maximum entropy model determined based on a training immune system repertoire that is known not to be associated with the at least one biological condition.

26. The computer system of claim 25, further comprising an all-model score generator configured to form, in an automated fashion by the processor, an all-model score classifier module implemented by the processor, the forming of the all-model score classifier module comprising determining with the processor a plurality of all-model scores against both the at least one biological condition-positive maximum entropy model and the at least one biological condition-negative maximum entropy model, the all-model classifier module permitting generating, in an automated fashion by the processor, a data structure representing at least one of: a histogram of the plurality of all-model scores versus a fraction of the test biological sequences, and a two or more dimensional cloud of the all-model scores.

27. The computer system of claim 26, wherein the all-model score generator is further configured to form the all-model score classifier by dividing, in an automated fashion by the processor, the plurality of scores against the at least one biological condition-positive maximum entropy model by the plurality of scores against the at least one biological condition-negative maximum entropy model, the dividing comprising desired weighting and normalizing.

28. The computer system of claim 26, wherein the classifier module is further configured to classify, in an automated fashion by the processor, the test immune system repertoire based on an increased probability density beyond expected probability density determined based on at least a portion of at least one of: the data structure representing the histogram of the plurality of all-model scores, and the data structure representing the two or more dimensional cloud of the all-model scores.

29. The computer system of claim 24, wherein the classifier module is further configured to classify the test immune system repertoire based on determining, in an automated fashion by the processor, a reduced subset of the bias parameters of the maximum entropy model that permit classifying the test immune system repertoire with a desired level of accuracy as being systematically associated with, or not systematically associated with, the at least one biological condition.

30-31. (canceled)

32. The computer system of claim 29, further comprising a maximum accuracy separator module configured to separate, in an automated fashion by the processor, at least one biological condition-positive maximum entropy model from at least one biological condition-negative maximum entropy model.

33. The computer system of claim 32, wherein the maximum accuracy separator module comprises a linear support-vector machine classifier.

34. The computer system of claim 24, wherein the at least one biophysical composite measure comprises a result of a dimensionality reduction of a plurality of individual amino acid measures.

35-42. (canceled)

43. The computer system of claim 24, wherein the classifier module further comprises a probability determination module configured to determine, in an automated fashion by the processor, a probability of the test immune system repertoire having been generated by the maximum entropy model.

44. The computer system of claim 24, wherein the classifier module is further configured to determine, in an automated fashion by the processor, similarity scores comparing at least two different test immune system repertoires with each other based on the maximum entropy model.

45. The computer system of claim 24, wherein the modeling module is configured to form the maximum entropy model by training, in an automated fashion by the processor, the maximum entropy model on the plurality of feature components using a Metropolis-Hastings Markov-Chain Monte-Carlo procedure.

46. A non-transitory computer-readable medium configured to store instructions for classifying an immune system repertoire, the instructions, when loaded and executed by a processor, cause the processor to classify the immune system repertoire by:
providing a data structure representing a plurality of training biological sequences that are included in at least one training immune system repertoire;
for the training biological sequences represented by the data structure, associating, in a manner automated by a processor, one or more biophysical properties and operatively indicating the biophysical properties in a plurality of training repertoire biophysical feature data structures;
the training repertoire biophysical feature data structures computationally representing the one or more biophysical properties of the training biological sequences based on expectation values of at least one biophysical composite measure for each of a plurality of feature components, the plurality of feature components including feature components corresponding to an amino acid sequence of the training biological sequences;
forming, in an automated fashion by the processor, a maximum entropy model based on the training repertoire biophysical feature data structures, the formed maximum entropy model comprising a bias parameter for each feature component of the plurality of feature components;
providing a data structure representing a plurality of test biological sequences that are included in at least one test immune system repertoire; and
based on the formed maximum entropy model and the data structure representing the plurality of test biological sequences, classifying, in an automated fashion by the processor, the test immune system repertoire, the classifying including classifying the test immune system repertoire as being associated with at least one biological condition or as not being associated with the at least one biological condition.
